(19) United States
(12) Patent Application Publication
(10) Pub. No.: US 2013/0331597 A1
(43) Pub. Date: Dec. 12, 2013

(54) AMIDATION PROCESS

(71) Applicants: Guy Humphrey, Hillsborough, NJ (US); Kelvin Yong, Lyndhurst, NJ (US)

(72) Inventors: Guy Humphrey, Hillsborough, NJ (US); Kelvin Yong, Lyndhurst, NJ (US)

(21) Appl. No.: 14/000,950

(22) PCT Filed: Feb. 27, 2012
(86) PCT No.: PCT/US12/26694

§ 371 Date: Aug. 22, 2013

Related U.S. Application Data
(60) Provisional application No. 61/448,412, filed on Mar. 2, 2011.

(51) Int. Cl. C07C 315/04

(52) U.S. Cl. CPC C07C 315/04
USPC 558/426

(57) ABSTRACT

This invention describes an amidation process whereby perfluorinated amino acids can be activated and treated with an amine in the presence of a coupling agent and a pyridine derivative to yield amides, without loss of optical purity.

### BACKGROUND OF THE INVENTION

This invention describes an amidation process whereby fluorinated amino acids can be activated and treated with an amine in the presence of a coupling agent and a pyridine derivative to yield amides, without loss of optical purity. The resulting amides are selective cathepsin K inhibitors which can be used in the treatment of osteoporosis and metastatic bone disease.

The art describes the amidation as including a coupling agent and a base, specifically HOBt. Dry HOBt is unstable and potentially explosive, so it would be desirable to develop an amidation process that does not utilize HOBt.

It would also be desirable to have an amidation process that is simpler, less expensive and uses readily available reagents; the instant process'meets those needs. Additionally, the instant process produces a higher yield when compared with previously known processes and can be run at lower temperatures.

### SUMMARY OF THE INVENTION

By this invention, there are provided processes for the preparation of compounds of structural formula I:

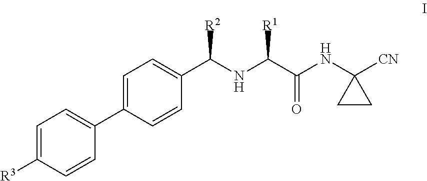

comprising amidating a salt of formula IIA or an acid of formula IIB

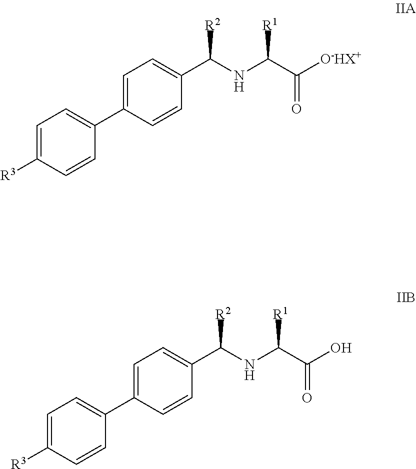

with 1-aminocyclopropane carbonitrile, in the presence of a coupling agent, a pyridine derivative and a solvent;
wherein R¹ is C₁₋₆ alkyl or C₁₋₆ haloalkyl;
R2 is C₁₋₆ alkyl or C₁₋₆ haloalkyl;
R³ is SOₘ(C₁₋₆ alkyl);
X is a tertiary amine, a secondary amine or a metal salt; and
m is an integer from zero to two.

### DETAILED DESCRIPTION OF THE INVENTION

By this invention, there are provided processes for the preparation of compounds of structural formula I:

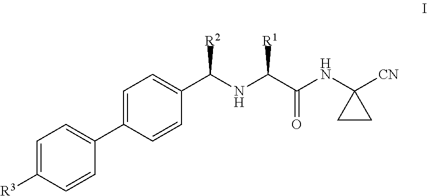

comprising amidating a salt of formula IIA or an acid of formula IIB

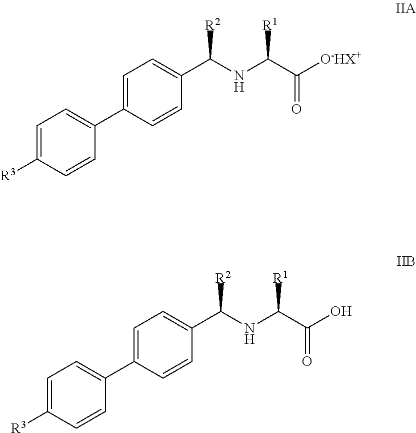

with 1-aminocyclopropane carbonitrile, in the presence of a coupling agent, a pyridine derivative and a solvent;
wherein R¹ is C₁₋₆ alkyl or C₁₋₆ haloalkyl;
R2 is C₁₋₆ alkyl or C₁₋₆ haloalkyl;
R3 is SOₘ(C₁₋₆ alkyl);
X is a tertiary amine, a secondary amine or a metal salt; and
m is an integer from zero to two.

In an embodiment of the invention, R¹ is C₁₋₆ haloalkyl. In a class of the invention, R¹ is (2-fluoro,2-methyl)propyl.

In an embodiment of the invention, R² is C₁₋₆ haloalkyl. In a class of the invention, R² is trifluoromethyl.

In an embodiment of the invention, R³ is SO₂(C₁₋₆ alkyl). In a class of the invention, R³ is SO₂CH₃.

In an embodiment of the invention X is a secondary amine. In a class of the invention, X is DCHA.

In an embodiment of the invention, the compound of formula I is

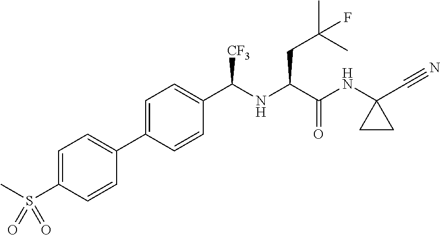 [SMILES: CC(C)(F)C[C@H](N[C@@H](c1ccc(-c2ccc(S(C)(=O)=O)cc2)cc1)C(F)(F)F)C(=O)NC1(C#N)CC1]

N¹-(1-cyanocyclopropyl)-4-fluoro-N²-{(1S )-2,2,2-trifluoro-1-[4′-(methylsulfanyl)-1,1′-biphenyl-4-yl]ethyl}-L-leucinamide.

An α-amino acid or its corresponding salt is combined with an amine in the presence of a coupling agent and a pyridine derivative. The reaction is run in the presence of a suitable solvent.

In an embodiment of the invention, the coupling agent is selected from the group consisting of a carbodiimide, phosphonium salt or uranium salt. In a class of the invention, the coupling agent is DCC, DIC, EDC, phosphonium iodide, tetramethylphosphonium iodide, PyBrOP, PyAOP, HBTU, HATU, TATU, TBTU and mixtures thereof. In a subclass of the invention, the coupling agent is EDC.

In an embodiment of the invention, the pyridine derivative is selected from the group consisting of pyridine, 4-phenylpyridine, 4-alkylpyridine, 3-alkylpyridine, 3,4-dialkylpyridine, 3-bromopyridine, 4-bromopyridine and mixtures thereof. In a class of the invention, the pyridine derivative is pyridine.

In an embodiment of the invention, the solvent is selected from the group consisting of DMF, DMAc, NMP, acetonitrile, THF, DMSO and mixtures thereof. In a class of the invention, the solvent is DMF.

In an embodiment of the invention is the process for the preparation of a compound of structural formula:

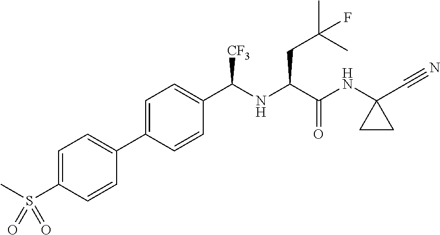 [SMILES: CC(C)(F)C[C@H](N[C@@H](c1ccc(-c2ccc(S(C)(=O)=O)cc2)cc1)C(F)(F)F)C(=O)NC1(C#N)CC1]

comprising amidating a salt:

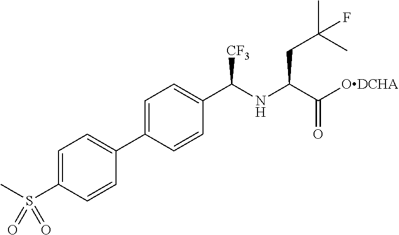

with 1-aminocyclopropane carbonitrile, in the presence of EDC, pyridine and DMF.

The term “alkyl” as used herein shall mean a substituting univalent group derived by conceptual removal of one hydrogen atom from a straight or branched-chain acyclic saturated hydrocarbon (i.e., —CH₃, —CH₂CH₃, —CH₂CH₂CH₃, —CH(CH₃)₂, —CH₂CH₂CH₂CH₃, —CH₂CH(CH₃)₂, —C(CH₃)_{3,} etc).

As appreciated by those of skill in the art, “halo” or “halogen” as used herein is intended to include chloro, fluoro, bromo and iodo. The term “keto” means carbonyl (CO═O). The term “alkoxy” as used herein means an alkyl portion, where alkyl is as defined above, connected to the remainder of the molecule via an oxygen atom. Examples of alkoxy include methoxy, ethoxy and the like.

The term “haloalkyl” means an alkyl radical as defined above, unless otherwise specified, that is substituted with one to five, preferably one to three halogen. Representative examples include, but are not limited to trifluoromethyl, dichloroethyl, and the like.

The term “tertiary amine” includes, but is not limited to, trimethylamine, triethylamine, tripropylamine, dimethylethanolamine and bis-tris.

The term “secondary amine” includes, but is not limited to, dimethyl amine, diethylamine, methylethanolamine, aziridine, azetidine, pyrrolidine, piperidine, and dicyclohexylamine (DCHA).

The term “metal salt” includes, but is not limited to, salts of aluminum, antimony, calcium, copper, gold, iron, lead, lithium, magnesium, platinum, potassium, sodium, silver, strontium, tin, titanium, tungsten and zinc. Preferred metal salts include salts of lithium, sodium, potassium, magnesium, calcium, aluminum and zinc.

The term carbodiimide refers to a class of coupling agents that are often used to activate carboxylic acids towards amide formation. Nonlimiting examples or carbodiimides include: DCC (N,N′-dicyclohexylcarbodiimide), DIC (N,N′-diisopropylcarbodiimide) and EDC (1-ethyl-3-(3-dimethylarninopropyl) carbodiimide hydrochloride).

The term phosphonium salt refers to salts containing the phosphonium ion ((PH₄⁺) ion) which are useful as coupling agents. Nonlimiting examples of phosphonium salts include: phosphonium iodide, tetramethylphosphonium iodide, PyBrOP (Bromo-tris-pyrrolidino phosphoniumhexafluorophosphate) and PyAOP ((7-Azabenzottiazol-1-yloxy)tripyrrolidinophosphonium hexafluorophosphate).

The term uronium salt refers to salts containing the uronium ion. Nonlimiting examples of uronium salts include: HBTU (2-(1H-Benzotriazole-1-yl)-1,1,3,3-tetramethyluronium hexafluorophosphate), HATU (2-(7-Aza-1H-benzotriazole-1-yl)-1,1,3,3-tetramethyluronium hexafluorophosphate), TATU ((O-(7-Azabenzotriazole-1-yl)-N,N,N′,N′-tetramethyluronium tetrafluoroborate) and TBTU (2-(1H-Benzotriazole-1-yl)-1,1,3,3-tetramethyluronium tetrafluoroborate).

In the schemes and examples below, various reagent symbols and abbreviations have the following meanings:

DMAc: N,N′-Dimethyl acetamide

DCHA: Dicyclohexylamine

MTBE: Methyl-t-butylether

iPAc: Isopropyl acetate

DMF: N,N′-Dimethylformamide

THF: Tetrahydrofuran

TEA: Triethylamine

DMSO: Dimethylsulfoxide

NMP: 1-Methyl-2-pyrrolidinone

CDI: N N′-Carbonyldiimidazole

HATU: O-(7-Azabenzotriazol-1-yl)-N,N,N′N′-tetramethyluronium hexafluorophosphate

EDC: 1-Ethyl-3-[3-dimethylaminopropyl]carbodiimide hydrochloride

HOBt: N-Hydroxybenzotriazole

NMM: N-methyl morphonline

Scheme 1 depicts the reaction of a substituted α-amino acid or its salt with an amine in the presence of a coupling agent, an activator and a base to yield the corresponding α-amino amide product without epimerization of the α-stereocenter.

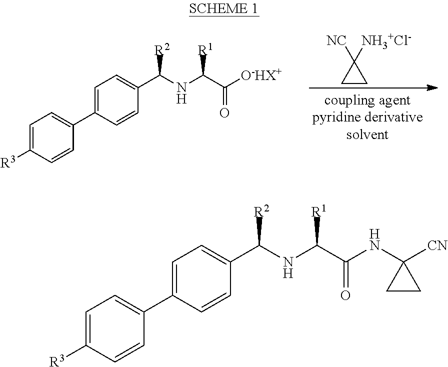

The following examples further illustrate details for the preparation of the compounds of the present invention. Those skilled in the art will readily understand that known variations of the conditions and processes of the following preparative procedures can be used to prepare these compounds. All temperatures are degrees Celsius unless otherwise noted.

### EXAMPLE 1

### 4-FLUORO-N-{(1S)-2,2,2-TRIFLUORO-1-[4′-(MTTHYLSULFONYL)BIPHENYL-4-YL]ETHYL}-L-LEUCINE DICYCLOHEXYLAMINE SALT

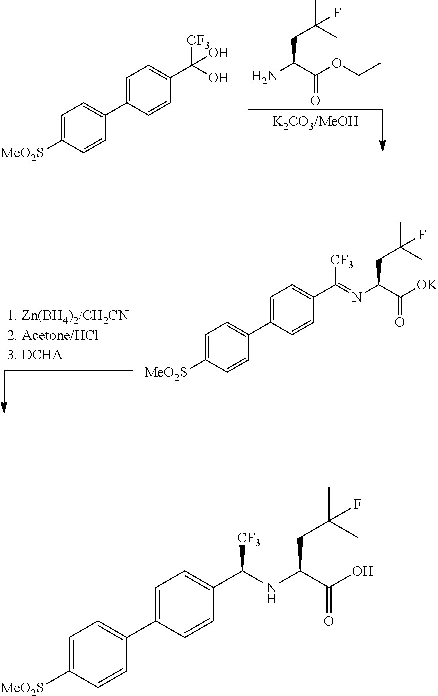

A 200 mL vessel was charged with 2,2,2-trifluoro-1[4′-(methylsulphonyl)biphenyl-4yl]ethane-1,1-diol (9.08 g, 26.2 mmol), F-leucine ethyl ester sulphate salt (8.66 g, 31.5 mmol), potassium carbonate (14.5 g, 104.9 mmol) and methanol (27.3 mL). The mixture was heated to 50° C., aged for 4 h and then cooled to −5° C.

A 500 mL vessel was charged with zinc chloride (7.15 g, 52.5 mmol) and dimethoxyethane (40.9 mL). The mixture was cooled to −10° C. and sodium borohydride (3.97 g, 104.9 mmol) charged in a portionwise manner. The mixture was aged at −10° C. for 1 h and acetonitrile (63.6 mL) added, maintaining the temperature below 0° C.

The imine mixture was then transferred to the borohydride solution, at such a rate as to maintain the temperature between −5 and +5° C. The reaction was then aged between −5 and +5° C. for 1.5 h, quenched by the slow addition of acetone (33.9 mL) and allowed to warm to 20° C. MTBE (60.6 mL), 2M HCl (181.7 mL) and DI Water (63.6 mL) were charged and the mixture aged for 30 min. The organic phase was separated and the aqueous re-extracted with MTBE (45.4 mL). The two MTBE phases were combined, washed with water (45.4 mL×4) and diluted with MTBE (139.3 mL). Dicyclohexylamine (5.23g, 28.8 mmol) was then charged over 30 min at 20° C. The product slurry was aged at 20° C. for 1 h, filtered and washed with TBME (36.3 mL). After drying in-vacuo at 30° C. to constant weight, the title compound was obtained as a white powder.

### EXAMPLE 2

### N-(1 -CYANOCYCLOPROPYL)-4-FLUORO-N²-{(1S)-2,2,2-TRIFLUORO-1-[4′-(METHYLSULFONYL) BIPHENYL-4-YL]ETHYL}-L-LEUCINAMIDE

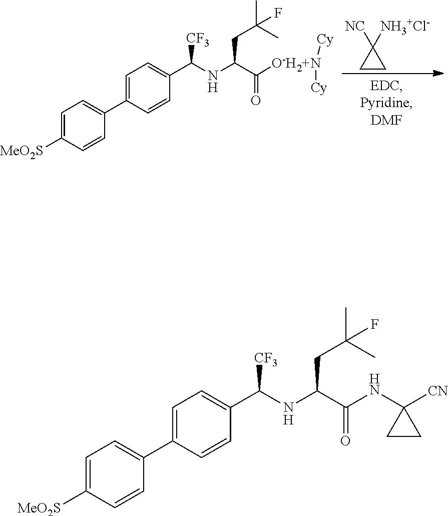

A round-bottom flask was charged with biphenyl acid.DCHA salt (10.3 g), aminocyclopropane carbonitrile.HCl (2.21 g), pyridine (2.46 g) and DMF (85 mL). The thick slurry was stirred at ambient temperature for 1 h. The slurry was cooled to −10° C. and EDC.HCl (4.47 g) added in one portion. The reaction mixture was aged at −10° C. for 1 h and warmed to −5° C. for 3 h. The batch was then warmed to 35° C. and aged 1 h. HPLC analysis showed 99.5% conversion. Aqueous phosphoric acid (100 mL) was added at 35-45° C. and the resultant slurry cooled to 20° C. The batch was filtered and washed with 55/45 DMF/water (50 mL) and water (50 mL). The solids were dried in the filter at 40-60° C. for 24 hours. The desired crude product was isolated as a white solid (7.57 g, 94% yield, >99% ee, 99.0 wt %). ¹H NMR (CD₃OD) δ8.17 (bs, 1H), 8.05 (d, 2H, J=8.5), 7.96 (d, 2H, J=8.5), 7.80 (d, 2H, J=8.0), 7.64 (d, 2H, J=8.0), 4.43 (m, 1H), 3.55 (ddd, 1H, J=5.0, 8.5, 8.0), 3.18 (s, 3H), 2.84 (bm, 1H), 2.02 (m, 2H), 1.46 (d, 3H, J=21.5), 1.43 (d, 3H, J=22.0), 1.36 (m, 2H), 1.07 (m,111), 0.94 (m, 1H); ¹³C NMR (CD₃OD) δ; ¹⁹F NMR (CD₃OD) δ−712, −136.8; IR (cm⁻¹) 3331, 2244, 1687, 1304, 1152; mp 223-224° C., [α]_{D}²⁰+23.3 (c=0.53, MeOH).

What is claimed is:

1. A process for preparing a compound of formula I: 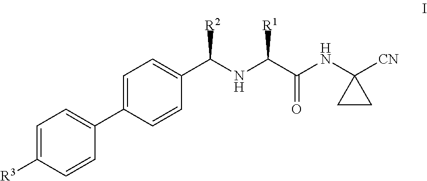
comprising amidating a salt of formula IIA or an acid of formula IIB 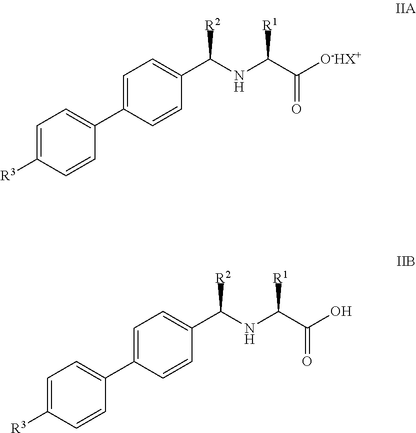
with 1-aminocyclopropane carbonitrile, in the presence of a coupling agent, a pyridine derivative and a solvent;
wherein R1 is C₁₋₆ alkyl or C₁₋₆ haloalkyl;
R² is C₁₋₆ alkyl or C₁₋₆ haloalkyl;
R³ is SOₘ(C₁₋₆ alkyl);
X is a tertiary amine, a secondary amine or a metal salt; and
m is an integer from zero to two.

2. The process of claim 1 wherein the pyridine derivative is selected from the group consisting of pyridine, 4-phenylpyridine, 4-alkylpyridine, 3-alkylpyridine, 3,4-dialkylpyridine, 3-bromopyridine, 4-bromopyridine and mixtures thereof.

3. The process of claim 2 wherein the pyridine derivative is pyridine.

4. The process of claim 1 wherein the solvent is selected from the group consisting of DMF, DMAc, NMP, acetonitrile, THF, DMSO and mixtures thereof.

5. The process of claim 4 wherein the solvent is DMF.

6. The process of claim 1 wherein the coupling agent is selected from the group consisting of a carbodiimide, phosphonium salt and uronium salt.

7. The process of claim 6 wherein the coupling agent is selected from the group consisting of DCC, DIC, EDC, phosphonium iodide, tetramethylphosphonium iodide, PyBrOP, PyAOP, HBTU, HATU, TATU, TBTU and mixtures thereof.

8. The process of claim 7 wherein the coupling agent is EDC.

9. The process of claim 1 wherein R¹ is C₁₋₆ haloalkyl; R² is C₁₋₆ haloalkyl and R³ is SO₂(C₁₋₆ alkyl).

10. The process of claim 6 wherein R¹ is (2-fluoro,2-methyl)propyl; R² is trifluoromethyl and R³ is SO₂CH₃.
